(19) United States
(12) Patent Application Publication
(10) Pub. No.: US 2024/0023285 A1
(43) Pub. Date: Jan. 18, 2024

(54) FLEXIBLE METAL CHIP COOLING INTERFACE

(71) Applicant: Quantum-Si Incorporated, Guilford, CT (US)

(72) Inventors: Todd Rearick, Cheshire, CT (US); Kyle Rearick, Cheshire, CT (US); Jonathan C. Schultz, Guilford, CT (US); Philip Trioli, New Boston, NH (US)

(21) Appl. No.: 17/864,370

(22) Filed: Jul. 13, 2022

(51) Int. Cl. H05K 7/20

(52) U.S. Cl. CPC H05K 7/20509; H05K 7/2049; G01N 33/53

(57) ABSTRACT

Heat transfer systems and methods are generally described. A bioanalytical instrument may employ a variety of heat-generating optoelectronic chips to perform biochemical assays. In some embodiments, the instrument may include a heat transfer system configured to cool the chips by thermally coupling them to a heat sink. The heat transfer system may include an elastically deformable flexible plate to conform to the differently-sized chips, withstand numerous cycles of chip insertion/removal, and respond to pressures exerted by supporting structures of the instrument. The heat transfer system may include a thermally conductive material indirectly but thermally coupled to the chip via the flexible plate, which may be positioned between the heat sink and the flexible plate.

### FIELD

The technology is generally related to bioanalytical instruments capable of performing massively parallel sample analyses and related methods. More specifically, aspects of the technology relate to systems and methods of thermal management for cooling heat-generating optoelectronic chips.

### BACKGROUND

Instruments that are capable of massively parallel analyses of biological or chemical specimens are typically limited to laboratory settings because of several factors, including a large volumetric footprint, lack of portability, requirement of a skilled technician to operate the instrument, power needs, need for a controlled operating environment, and excessive cost. When a sample is to be analyzed using such equipment, a common paradigm is to extract a sample at a point of care or in the field, send the sample to the lab, and wait for results of the analysis. The wait time for results can range from hours to days.

### SUMMARY

The technology described herein relates to a bioanalytical instrument for massively parallel sample analyses. The instrument may be useful for clinical and/or point-of-care sample analysis such as nucleic acid or protein sequencing or for personalized medicine. The instrument can be used for other applications (e.g., drug or protein detection, virus detection, tracking of virus or bacteria mutations, proteomics, and metabolic assays) that involve analyses of samples.

According to some aspects, heat transfer systems are provided. The heat transfer system includes a plate configured to deform to receive an optoelectronic chip, wherein the plate is configured to be in thermal communication with the optoelectronic chip at a first interface, and wherein the plate is in thermal communication with a heat sink via a thermally conductive material.

According to some aspects, methods of cooling an optoelectronic chip are provided. The method includes deforming a plate to receive the optoelectronic chip on a first surface of the plate, such that the plate and the optoelectronic chip are in thermal communication; transferring to the plate heat from the optoelectronic chip; and transferring to a thermally conductive material positioned on an opposing surface of the plate heat from the plate.

According to some aspects, methods of manufacturing a heat transfer system are provided. The method includes coupling at least a portion of a first surface of a plate to at least a portion of a first surface of a housing to form a reservoir therebetween; and introducing a thermally conductive material into the reservoir through at least one port formed in the housing; wherein the thermally conductive material is in thermal communication with the plate and the housing, and wherein the housing is in thermal communication with a heat sink.

According to some aspects, heat transfer systems are provided. The heat transfer system includes a thermal interface configured to undergo substantially elastic deformation to receive a chip, wherein the thermal interface is configured to be in thermal communication with the chip, and wherein the thermal interface is configured to repeatedly receive and release one or more chips.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. The skilled artisan will understand that the figures, described herein, are for illustration purposes only. It is to be understood that in some instances various aspects of the invention may be shown exaggerated or enlarged to facilitate an understanding of the invention. The drawings are not necessarily to scale, emphasis instead being placed upon illustrating the principles of the teachings. The drawings are not intended to limit the scope of the present teachings in any way. In the drawings:

FIG. 1 is a perspective view of an assembled heat transfer system, according to some embodiments;

FIG. 2 is a perspective exploded view of a heat transfer system, according to some embodiments;

FIG. 3 shows, according to some embodiments, a method of assembly of a heat transfer system;

FIG. 4 is a perspective exploded view of another embodiment of a heat transfer system;

FIGS. 5A-5C are partial cross-sectional views of the heat transfer system of FIG. 1 taken along A-A;

FIG. 6 is a top view of a flexible plate of the heat transfer system, according to some embodiments; and

FIGS. 7A-7C are various views of another embodiment of a flexible plate.

### DETAILED DESCRIPTION

The Inventors have recognized that an instrument capable of massively parallel sample analyses may be highly useful for rapid testing services such as specimen analysis (e.g., sensing for harmful gaseous leaks, combustion by-products, or toxic chemical components, etc.), patient specimen analysis (e.g., blood, urine, etc.) or nucleic acid or protein sequencing. In some cases, it is desirable to have portable, hand-held instruments for analyzing samples, so that technicians or medical personnel can easily carry the instrument into the field to rapidly and accurately analyze a given sample. In clinical settings, a desk-top size instrument may be desired for more complex sample analysis.

In addition, the Inventors have recognized that such an instrument may be able to achieve massively (e.g., tens or hundreds of thousands) parallel testing and analysis by integrating a number of sample wells or reaction chambers on a consumable chip, which may be received by the instrument. The Inventors have recognized that the chip may benefit from features such as integrated optics (e.g., to uniformly excite/illuminate each reaction chamber) and photodetectors (e.g., to detect emissions from the reaction chambers) to reduce a large number of external optical components that might otherwise be needed. An instrument may interact with hundreds or thousands of consumable chips during its lifetime, with each chip having slight variations in geometry.

The Inventors have recognized that such a multifunctional chip and associated electronics may generate heat during operation. Accordingly, it may be necessary to dissipate said heat to reduce the likelihood of adversely affecting the performance, reliability, and lifetime of the chip and other components (e.g., optical components or biological materials) contained within the instrument. For example, the temperature of the chip may need to be controlled to ensure that reactions within the sample wells proceed with a low likelihood of specimens (such as genomic or proteomic materials) degrading while undergoing testing within the instrument.

Heat-generating elements can be cooled using heat sinks configured to rapidly transport heat away from the element to maintain said element at a desired temperature. For example, heat sinks are used in microelectronics devices as a means of thermal management. Heat sinks can be passive, such as heat pipes or high surface area/high thermal conductivity metallic bodies, or active, such as thermo-electric coolers (hereinafter referred to as “coolers”) or fans. To optimize heat flow away from the heat-generating element, a thermally conductive material, such as a thermal compound (with a thermal conductivity greater than air) can sometimes be applied in between the element and the heat sink to thermally couple the two components and reduce the thermal resistance of the interface.

The Inventors have recognized that employing conventional chip cooling technologies (e.g., applying a thermal compound such as thermal paste to thermally couple a heat sink to a heat-generating element) to cool a consumable chip used in a bioanalytical instrument may be challenging and impractical given the significant number of variably sized chips any individual instrument may interact with. Specifically, hundreds and thousands of chips may be installed and removed from the instrument during its lifetime. Accordingly, applying a thermal compound to each individual chip of hundreds and thousands of chips inserted into the instrument can be time-consuming, costly, and lead to undesirable contamination. For example, the Inventors have recognized that the process of precisely applying a thermal compound to a surface of a chip, inserting said chip into the instrument, removing said chip from the instrument, and cleaning the thermal compound off of the chip and the instrument following every test can be impractical and wasteful with regards to material (e.g., thermal compound) and time. In addition, repeated application and removal of the thermal compound may lead to contamination of the instrument. In some cases, contamination of the instrument with the thermal compound may result in instrument damage and/or dysfunction.

In some instances, a consumable chip may be inserted into and removed from a bioanalytical instrument through one or more supporting structures (e.g., a clamping system for accepting/releasing the chips) to ensure proper and consistent positioning of the chip in the instrument after insertion and during testing. The supporting structures may exert pressures on the chips within the instrument, such that portions of the chip in contact with the supporting structures may undergo deformation. The Inventors have also recognized that such deformations may reduce the quality of contact between the chip and a heat sink of the bioanalytical instrument, resulting in greater thermal resistance in between the heat sink and the chip. In this way, the deformation can reduce the cooling efficiency of the heat sink, and can, in some instances, result in overheating of the chip.

In view of the above, the Inventors have recognized the benefits of an apparatus and associated methods for making and operating a bioanalytical instrument capable of repeated, massively parallel sample analyses. In overview and according to some embodiments, the instrument may include a heat transfer system for an optoelectronic chip (hereinafter referred to as a “chip”). In particular, the Inventors have recognized the benefits of a heat transfer system that can effectively cool a variety of differently sized consumable chips to biologically-useful temperatures, by thermally coupling said chips to a heat sink. In addition, as noted previously, chip deformation due to the use of supporting or clamping structures may lead to inefficient cooling due to reduced quality of contact between the chip and the heat sink. Repeated insertion and removal cycles of a consumable chip from a bioanalytical instrument may also deform the chip, resulting in inconsistent heating/cooling between cycles. Thus, the Inventors have also recognized the benefits of a heat transfer system that is convenient to use and can consistently cool the chip spanning a significant number of chip insertion and removal cycles by maintaining a consistent thermal resistance in between the chip and the heat sink—regardless of deformation and/or the frequency of chip insertion/removal from the instrument. Of course, instances in which different benefits are offered by the systems and methods disclosed herein are also possible.

According to exemplary embodiments described herein, a bioanalytical instrument may include a heat-generating element, such as an optoelectronic chip, and a heat transfer system configured to cool said element. In some embodiments, the heat transfer systems described herein may include a heat sink, which may be an active component, such as a thermo-electric cooler (e.g., a Peltier device). To accommodate slight variations in chip geometries, repeated insertion/removal of said chips into the bioanalytical instrument, and pressures exerted by supporting structures within the instrument (e.g., clamping systems) designed to receive and maintain the chips in a constant position, the heat transfer systems described herein may employ a flexible plate in thermal communication with the chip and the heat sink. In some embodiments, the flexible plate may elastically deform to receive and conform to a variety of differently sized chips and/or in response to pressures exerted by supporting structures of the instrument. Conformal contact between the plate and chip may reduce the thermal resistance at the interface of said components when compared to a flat, inflexible plate.

In some embodiments, a heat transfer system may include a flexible plate configured to elastically deform. The elastic deformation of the plate may serve multiple purposes, including, but not limited to, to receive a variety of differently sized optoelectronic chips, to respond to pressures exerted by supporting structures (e.g., a clamping system for accepting/releasing the chips) of the instrument, and/or to accommodate a significant number of insertion/removal cycles of the chips. The heat transfer system may also include a heat sink (e.g., a cooler) and optionally a thermally conductive material (e.g., a thermal compound with a thermal conductivity greater than air) located in between the plate and heat sink. The flexible plate may be configured to be in contact with the optoelectronic chips on one surface (e.g., an optoelectronic chip may press against the surface of the flexible plate when installed), and in contact with the thermal compound on an opposing surface. The thermal compound may then be in direct or indirect contact with the heat sink. In this way, the optoelectronic chips may be in thermal communication with the heat sink through the thermal compound and the flexible plate. Thus, in some embodiments, the heat transfer system is configured such that thermal compound is not required to be placed between the flexible plate and an optoelectronic chip when installed. In some embodiments, the heat sink may be positioned in a heat sink housing, such that the thermal compound may be in thermal communication with the heat sink through the heat sink housing. As discussed previously, the thermal compound (which may, in some embodiments, be a thermal paste, thermal grease, thermal gel, thermal oil, thermal pad, combinations thereof, etc.) may reduce the thermal resistance of the interface at which it may be applied.

Heat transfer systems described herein may operate to transport heat away from an optoelectronic chip, which may generate heat when carrying out one or more functions (e.g., biochemical assays). The heat transfer system may be part of a thermal circuit including a chip, an optional chip interface module, a flexible plate, a thermal compound, a heat sink housing, and the heat sink itself. In some embodiments, an optional chip interface module may be positioned in between the chip and the flexible plate. The module may include a printed circuit board and electronic components (e.g., integrated circuit chips and discrete components such as resistors, capacitors, diodes, transistors, inductors, etc.) to enable various optoelectronic functions of the chip.

In some embodiments, a method of assembly of a heat transfer system may include attaching a pre-formed flexible plate to a heat sink housing using one or more binders. In some embodiments, the one or more binders may effectively seal off an internal volume or reservoir formed in between the heat sink housing and plate, save for one or more conduits configured to be fluidically connected to a surrounding environment. The conduits (e.g., channels connected to ports) may be used to introduce a thermal compound into the reservoir, and may also allow air and/or the thermal compound to flow out of the reservoir upon deformation of the flexible plate.

The one or more binders may comprise any suitable material configured to attach the plate to the heat sink housing. In some embodiments, the binder may be stable at the operational temperatures of the heat transfer system and may absorb mechanical stresses from the cyclical loading/unloading of the chip/chip interface module without significant loss in structural integrity and/or adhesive properties. In some embodiments, the binder may be an adhesive, epoxy, solder, combinations thereof, and/or any other suitable substance that can robustly attach the flexible plate to the heat sink housing. In some embodiments, the binder may join the plate and the housing using a conductive solder and associated solder reflow processes. In this way, the binder may enhance the overall thermal conductivity of the heat transfer system (e.g., by allowing heat to flow out of the plate through the binder and the thermal compound, instead of primarily through the thermal compound) as well as the mechanical resilience of the interface between the plate and housing. It should be appreciated that any suitable material or combination of materials may be used to join the housing and flexible plate, as the present disclosure is not so limited.

In some embodiments, a thermal compound may be introduced into a reservoir in between the flexible plate and the heat sink housing prior to operation. Accordingly, the thermal compound may reduce the thermal resistance of said interface, facilitating more efficient heat transfer away from the chip. The thermal compound may directly contact a bottom surface of the flexible plate, and may therefore be configured to deform along with the flexible plate to maintain consistent thermal contact between the plate and housing. In other words, the thermal compound may flow and/or deform in between the plate and housing (e.g., in a designated reservoir) to accommodate the changing profile of the flexible plate. In this way, the thermal compound may thermally couple the plate and housing (and subsequently, the heat sink) regardless of the conformation of the flexible plate.

In order to conform to the deforming profile of the flexible plate and maintain good thermal contact between the plate and housing, the thermal compound may be flowable and thermally conductive. The thermal compound may be sufficiently viscous to avoid undesirable flow (e.g., outflow of thermal compound from the interface when the chip and/or chip interface is loaded on the flexible plate), but may still deform along with the flexible plate in order to maintain contact with the plate during operation. In some embodiments, the thermal compound may be injectable, either by having a sufficiently low viscosity such that it can be pressurized and extruded (e.g., through a syringe), and/or by having shear-thinning properties. In some embodiments, the thermal compound may be inert in air or water (e.g., may not swell or degrade upon exposure to air or water), and may be stable at the operational temperatures of the heat transfer system. In some embodiments, the thermal compound may have sufficient compliance and viscosity to absorb any impact or stress from the flexible plate (e.g., when a chip/chip interface module is installed or removed) without transmitting said loads to the heat sink housing. It should be appreciated that the present disclosure is not limited by the properties of the thermal compound.

In some embodiments, the thermal compound may be formed of a carrier and a thermally conductive powder. The carrier material may be any suitable fluid, flowable material, injectable material or matrix, which may be conductive or non-conductive, as the present disclosure is not so limited. In some embodiments, the carrier may include silicone grease, polydimethylsiloxane, methyl silicone, liquid-phase rubber, urethanes, acrylates, copolymers thereof, combinations thereof, and/or any other suitable carrier material. In some embodiments, the thermally conductive powder may include aluminum oxide, magnesium oxide, aluminum nitride, boron nitride, zinc oxide, monocrystalline diamond, graphite, ceramics, metals (including, but not limited to, copper, silver, aluminum, gold, tin, zinc, nickel, iron, and lead), metal alloys (e.g., stainless steel), high thermal conductivity nanomaterials (e.g., carbon nanotubes), combinations thereof, and/or any other suitable conductive material. In some embodiments, the thermal compound may include a mixture of one or more carriers and one or more conductive powders. It should be appreciated that any suitable thermal compound known to one of skill in the art may be employed to reduce the thermal resistance between the flexible plate and the heat sink housing, as the present disclosure is not so limited.

In some embodiments, the thermal compound may be introduced into a pre-assembled heat transfer system through a port or inlet formed in the heat sink housing (or any other suitable component of the heat transfer system). The port may be fluidically connected to a reservoir positioned in between the plate and the heat sink housing, such that introducing (e.g., through injection) the thermal compound into the reservoir may thermally couple the plate and the heat sink housing. In some embodiments, the heat sink housing (or any other suitable component of the heat transfer system) may include more than one port, such that at least one port may be used to introduce the thermal compound into the reservoir, and at least one port may be used as a vent. The vent may allow fluid (e.g., air or other gases within the reservoir, thermal compound, combinations thereof) to flow out of the reservoir to accommodate for the changing spatial volume of the reservoir when the flexible plate is deformed, as will be described in greater detail below. In some embodiments, one or more ports may be closed after introduction of the thermal compound into the reservoir with a closure (e.g., a set screw) to reduce the likelihood of leakage. It should be appreciated that any number of ports, closures, channels connecting ports to the reservoir, and/or any other suitable fluidic infrastructure may be employed, as the present disclosure is not so limited.

In some embodiments, a heat transfer system (including a flexible plate, a thermal compound, a heat sink housing, and a heat sink) may maintain a chip at a particular temperature. For example, in embodiments where the optoelectronic chip includes biological materials (e.g., proteins, enzymes, nucleic acid molecules), the chip may be kept at suitable physiological or biologically-useful temperatures to reduce the likelihood of degradation, denaturation, desorption, and/or any undesirable chemical reaction which may occur at elevated temperatures.

The heat transfer system may include one or more components (e.g., the conductive but flexible plate, the thermal compound, the heat sink housing, etc.) which work to maintain low thermal resistance and at least one heat sink, which may keep the chip at the desired temperature (e.g., below 27° C.) or range of temperatures. In some embodiments, the heat transfer system may maintain the chip at least at 15° C., 20° C., 25° C., 27° C., 28° C., 30° C., 32° C., 35° C., 37° C., 40° C., 42° C., 45° C., 50° C., and/or any other suitable temperature. In some embodiments, the heat transfer system may maintain the chip at less than equal to 50° C., 45° C., 42° C., 40° C., 37° C., 35° C., 32° C., 30° C., 28° C., 27° C., 25° C., 20° C., 15° C., and/or any other suitable temperature. Combinations of the foregoing ranges are also contemplated, including between 15° C. and 50° C., 20° C. and 40° C., 20° C. and 27° C., 20° C. and 37° C., 25° C. and 37° C., 27° C. and 37° C., and/or any other suitable range of temperatures. It should be appreciated that the properties of any component of the thermal circuit (including the heat transfer system) may be selected to cool a chip to a suitable temperature corresponding to a desired biological assay. Accordingly, the present disclosure is not limited by the temperature of the chip.

In some embodiments, the temperature of the chip may be directly or indirectly measured using one or more sensors positioned along the thermal circuit. For example, the heat sink housing may employ a sensor (e.g., a thermistor) to measure the temperature of the heat sink. The chip may also include one or more temperature sensors to evaluate the effectiveness of the heat transfer system in cooling the chip. In some embodiments, the heat transfer system may include a processor and/or controller capable of receiving temperature data from the one or more sensors of the thermal circuit and adjusting the cooling parameters (e.g., adjusting input current to the heat sink to adjust the heat sink's performance) to control the temperature of the chip.

In order to effectively cool the chip to a desired temperature, the thermal circuit's thermal resistance may be sufficiently low to enable continuous heat flow away from the chip. Each interface between the various components of the thermal circuit (the chip, the chip interface module, the flexible plate, the thermal compound, the heat sink housing, and the heat sink) may have a thermal resistance. Accordingly, the overall thermal resistance of the thermal circuit may be evaluated with the sum of each interface's thermal resistance. In some embodiments, the thermal resistance of the thermal circuit may be 1 K/W. In some embodiments, the thermal resistance of the thermal circuit may be at least 0.05 K/W, 0.1 K/W, 0.2 K/W, 0.5 K/w, 0.7 K/W, 0.8 K/W, 1 K/W, 1.2 K/W, 1.5 K/W, 2 K/W, 3 K/W, 4 K/W, 5 K/W, and/or any other suitable thermal resistance. In some embodiments, the thermal resistance of the thermal circuit may be less than or equal to 5 K/W, 4 K/W, 3 K/W, 2 K/W, 1.5 K/W, 1.2 K/W, 1 K/W, 0.8 K/W, 0.7 K/W, 0.5 K/W, 0.2 K/W, 0.1 K/W, 0.05 K/W, and/or any other suitable thermal resistance. Combinations of the foregoing ranges are also contemplated, including between 0.05 K/W and 5 K/W, between 0.5 K/W and 1.5 K/W, and/or any other suitable range of thermal resistance. It should be appreciated that the present disclosure is not limited by the thermal resistance of the thermal circuit and/or any specific interface of the thermal circuit.

In some embodiments, the thermal resistance of the interface between the chip or chip interface module and the flexible plate may be the largest resistance of the thermal circuit. In some embodiments, elastic deformation of the flexible plate (e.g., to accommodate various chip or chip interface module sizes, pressures exerted by support structures of the instrument, and/or repeated insertion/removal cycles of the chip/chip interface modules) may reduce this resistance, minimizing the volume of insulating air (i.e., the air gap) in between the two components. In other words, improved contact between the chip or chip interface module and the flexible plate may reduce the thermal resistance of said interface. In some embodiments, the thermal resistance of the interface between the chip or chip interface module and flexible plate may be 0.5 K/W or any other suitable thermal resistance (e.g., equal to, less than, or greater than the thermal resistances noted above with respect to the thermal resistance of the thermal circuit). In some embodiments, the thermal resistance of the interface between the chip or chip interface module and the flexible plate may be less than the thermal resistance of said interface if a non-flexible plate were used (which may include an air gap in between the chip and the non-flexible plate).

It should be appreciated that the flexible plate may be flat or any other suitable shape, as the present disclosure is not so limited. In some embodiments, the flexible plate may include one or more flat portions and/or one or more non-flat portions. In other words, the term “plate” as used herein is not limited to a two-dimensional and/or planar structure.

In some embodiments, the heat transfer system (including the flexible plate, thermal compound, heat sink housing, and heat sink) may cool the chip by heat dissipation. In some embodiments, the heat transfer system may dissipate 10 W of heat to maintain the chip at a desired temperature. In some embodiments, the heat transfer system may dissipate at least 1 W, 2 W, 3 W, 5 W, 8 W, 10 W, 12 W, 15 W, 20 W, and/or any other suitable heat load. In some embodiments, the heat transfer system may dissipate less than or equal to 20 W, 15 W, 12 W, 10 W, 8 W, 5 W, 3 W, 2 W, 1 W, and/or any other suitable heat load. Combinations of the foregoing ranges are also contemplated, including, but not limited to, 1 W to 20 W, 5 W to 20 W, 2 W to 20 W, and/or any other range of suitable heat loads. It should be appreciated that the present disclosure is not limited by the heat loading of the heat transfer system.

In some embodiments, the flexible plate may facilitate heat transfer away from a chip and any associated chip interface modules. Accordingly, the flexible plate may be formed of a thermally conductive material.

In some embodiments, the flexible plate may be elastically deformed over multiple cycles as a user loads or inserts a chip (and/or chip interface module), unloads said chip, and loads another chip. Accordingly, the flexible plate may be resilient, such that it may undergo multiple cycles without significant wear or degradation in elasticity. In some embodiments, the flexible plate may undergo hundreds, thousands, tens of thousands, hundreds of thousands, and/or any other suitable number of cycles of elastic deformation. It should be appreciated that each elastic deformation cycle may require a different magnitude of elastic deformation. For example, a user may insert a first chip, remove said chip, insert a second chip, which may be slightly larger than the first chip, remove said chip, and insert a third chip, which may be slightly smaller than the first chip. The flexible plate of the present disclosure may withstand variable elastic deformation over any suitable number of cycles without significant wear or degradation, while maintaining desirable conformational contact between the plate and the chip in every cycle. Accordingly, the flexible plate may be formed of any suitable ductile and resilient material capable of undergoing repeated elastic deformation to accommodate repeated insertion/removal cycles of a variety of differently sized chips or associated chip interfaces without significant plastic wear. In addition, the plate may be sufficiently flexible to respond to compressive or flexural forces exerted by supporting structures of the instrument (e.g., a clamping system for accepting/releasing the chips).

In some embodiments, the flexible plate may be formed using one or more manufacturing techniques, including, but not limited to, casting, stamping, forging, drawing, bending, coining, rolling, drilling, turning, grinding, welding, soldering, machining, plating, coating, spraying, work-hardening, polishing, rolling, and/or any other suitable technique. Accordingly, the flexible plate may be formed of any suitable material which may be compatible with the desired manufacturing technique. In some embodiments, the flexible plate may be formed of and/or may include a coating consisting of a corrosion-resistant material to reduce the likelihood of corrosion in case of exposure to fluid (e.g., condensation or accidental leakage) or humidity.

In some embodiments, the flexible plate may be formed of a material with high thermal conductivity, elasticity, resilience, formability, and/or any other desirable material property. In some embodiments, the flexible plate may be formed of aluminum, copper, stainless steel, silver, lead, tin, gold, brass, nickel, titanium, alloys thereof, conductive polymers or copolymers, conductive ceramics, composites, combinations thereof, and/or any other suitable material. It should be appreciated that the present disclosure is not limited by the material composition of the flexible plate.

In some embodiments, the flexible plate may be formed from sheet metal with a stamping and/or coining process. For example, a portion of a metal sheet may be coined (e.g., may undergo plastic flow as a result of stamping) to yield a smooth and flexibly deformable surface on the plate. In some embodiments, the starting sheet metal may be 2 gauge, 5 gauge, 6 gauge, 7 gauge, 8 gauge, 9 gauge, 10 gauge, 11 gauge, 12 gauge, 13 gauge, 14 gauge, 15 gauge, 16 gauge, 17 gauge, 18 gauge, 19 gauge, 20 gauge, 21 gauge, 22 gauge, 23 gauge, 24 gauge, 25 gauge, 26 gauge, 27 gauge, 28 gauge, 29 gauge, 30 gauge, 31 gauge, and/or any other suitable thickness, as the present disclosure is not limited by the thickness of the sheet metal used to form the flexible plate. In some embodiments, the manufacturing process (e.g., coining) may further reduce the thickness of a portion of the plate. It should be appreciated that the flexibility of the plate may depend on material properties (e.g., elastic modulus) as well as geometric properties (e.g., thickness of the flexible portion). Accordingly, in some embodiments, reducing the thickness of at least a portion of the plate may enhance the bending elasticity of the said portion based on plate bending theory.

In some embodiments, the elastic deformation of the flexible plate (or at least a portion of the flexible plate) may be quantified by strain, measured by the deflection of the plate when receiving a chip/chip interface module. Accordingly, the flexible portion of the plate may undergo any suitable magnitude of strain corresponding to elastic deformation to conform to a variety of differently sized chips/chip interface modules, repeated insertion/removal of chips/chip interface modules, and/or pressures exerted by supporting structures of the instrument. In some embodiments, at least a portion of the flexible plate may undergo at least 0.0005%, 0.002%, 0.005%, 0.01%, 0.02%, 0.05%, 0.07%, 0.1%, 0.2%, 0.3%, 0.4%, 0.5%, 0.7%, 1%, 2%, 5%, 10%, and/or any other suitable strain. In some embodiments, at least a portion of the flexible plate may undergo less than or equal to 10%, 5%, 2%, 1%, 0.7%, 0.5%, 0.4%, 0.3%, 0.1%, 0.07%, 0.05%, 0.02%, 0.01%, 0.005%, 0.002%, 0.001%, 0.0005%, and/or any other suitable strain. Combinations of the foregoing ranges are also contemplated, including, but not limited to, between 0.005% and 10%, 0.01% and 0.4%, and/or any other suitable strain ranges. It should be appreciated that the plate may undergo any number of variable strains and over any number of cycles. In some embodiments, the plate may deform to a fraction (e.g., half, one-fourth, one-tenth, one-hundredth, etc.) of its maximal strain capability to accommodate a chip/chip interface module.

In some embodiments, one or more surfaces of the flexible plate (or any other component of the heat transfer systems described herein) may undergo surface finishing to enhance certain surface properties. For example, the flexible plate may be plated with nickel and/or may include a nickel layer (e.g., through an electrolytic or electroless plating process) to improve adhesion with the binder. In some embodiments, nickel plating may also improve ductility and corrosion-resistance of the plate.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein. For example, although a heat transfer system for cooling an optoelectronic chip has been described, the heat transfer system described herein may be employed to cool any suitable heat-generating system. Furthermore, the heat transfer system described herein may be employed to maintain any suitable interface at a given temperature (e.g., physiological or biologically-useful temperature).

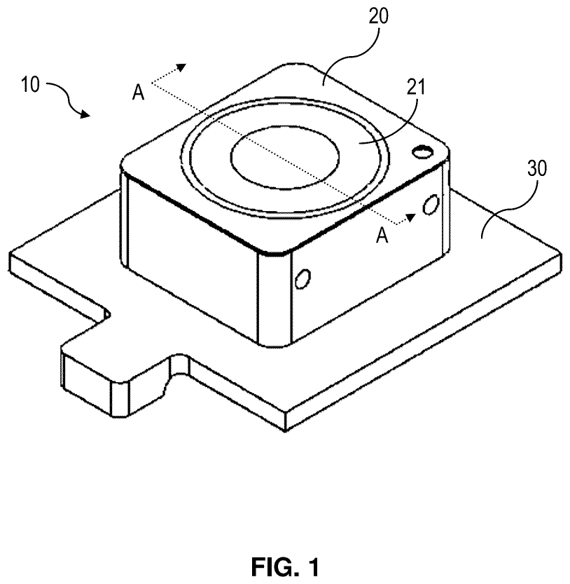

FIG. 1 shows an assembled heat transfer system 10 according to some embodiments. The heat transfer system 10 may include a flexible plate 20 installed on a housing containing a heat sink. As described above, the housing 30 may contain a thermo-electric cooler, or any other suitable heat sink (active, passive, or combinations thereof), as the present disclosure is not so limited. In some embodiments, a volume of thermal compound may be positioned in between the flexible plate 20 and housing 30 to reduce the thermal resistance in between the two components.

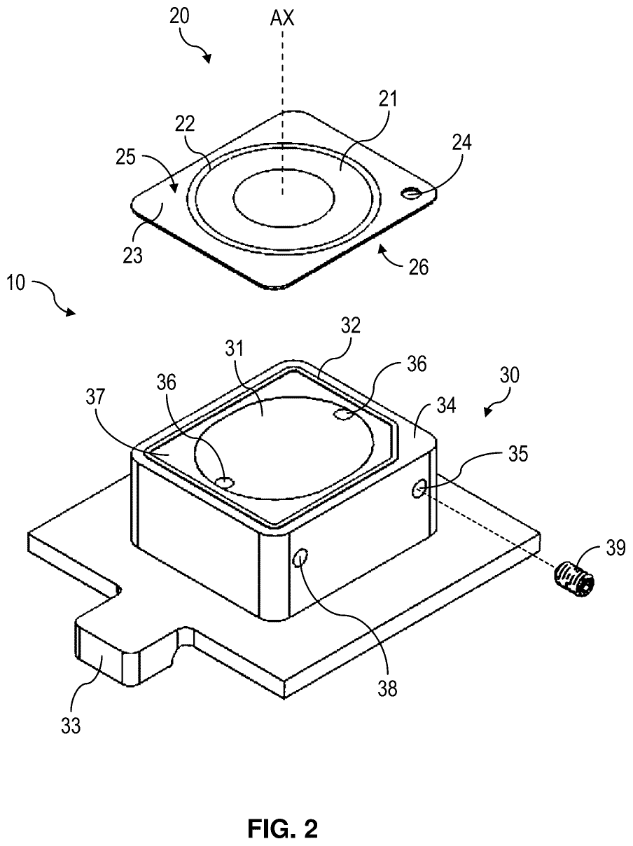

FIG. 2 shows an exploded view of a heat transfer system 10 according to some embodiments. The flexible plate 20 may include a surface 21 configured to elastically deform along an axis AX to receive a chip or chip module interface, as shown in FIG. 2. In some embodiments, the surface 21 may undergo in-plane deformation with respect to the plate 20. In some embodiments, bending of the flexible plate 20 may allow the plate 20 to better conform to a surface of the chip. The surface 21 may be formed by any suitable manufacturing technique, such as, for example, coining. Accordingly, the surface 21 may be thinner than a surrounding surface 23, which may not have been deformed during the coining process, and may accordingly be less flexible than the surface 21. The plate 20 may further include a periphery 22 in between the surfaces 21 and 23. In some embodiments, the periphery 22 may be a remnant of the manufacturing technique used to form the surface 21. The periphery 22 may improve the overall resilience of the plate 20.

In some embodiments, the plate 20 may include a top surface 25 configured to contact a chip or related chip module, and a bottom surface 26 configured to contact the housing 30. In some embodiments, the plate 20 may include an alignment marker 24 to facilitate alignment between the plate 20 and the housing 30. Accordingly, the housing 30 may include a surface 34 configured to facilitate alignment with the marker 24. In some embodiments, the marker 24 may be a through hole (as shown in FIG. 2), through which the surface 34 may be visible. The surface 34 may be visually distinct from the remainder of the housing (e.g., may be a rounded edge), such that a user may readily be able to arrange the plate 20 to align with the housing 30. It should be appreciated that any suitable arrangement of the marker 24 and surface 34 may be employed to allow a user to accurately place the plate 20 on the housing 30, as the present disclosure is not so limited.

In some embodiments, the housing 30 may include at least one tab 33, which may support one or more sensors. For example, tab 33 may support a thermistor configured to measure the temperature of the heat sink contained within the housing 30. In some embodiments, the housing may include one or more processors and/or controllers configured to communicate with sensors of the heat transfer system (or more broadly, sensors of the bioanalytic instrument) to better control the cooling of the chip.

In some embodiments, the housing 30 may include an upper surface 37 surrounding a reservoir 31. The reservoir 31 may be in thermal communication with a heat sink (e.g., a cooler) contained within the housing 30 (not shown). In some embodiments, the reservoir 31 may be in fluid communication with ports 35 and 38 through channels 36. For example, a fluid (e.g., a thermal compound) may be introduced into the reservoir 31 through port 35, such that it may flow through at least one channel 36 to reach the reservoir 31. The fluid (or any other substance, e.g., air) may then escape out of the reservoir 31 through at least one channel 36 and port 38. It should be appreciated that although two distinct channels 36 and two ports 35 and 38 are depicted in FIG. 2, any suitable number of channels/ports may be employed, as the present disclosure is not so limited. For example, in some embodiments, a heat sink housing may include at least two outlet ports (similar to port 38 of FIG. 2) positioned across the reservoir 31, such that fluid may readily flow out of the reservoir 31. In some embodiments, at least one port (e.g., port 35) may be blocked with a closure 39 (e.g., a set screw), such that fluid flow through the port 35 may be inhibited, and fluid flow through port 38 may be encouraged. As will be described in greater detail below, the port 38 may allow air to flow into and out of the reservoir 31 when the plate 20 is deformed about the axis AX.

As shown in FIG. 2, in some embodiments, the reservoir 31 may be arranged as a depression along a top surface of the housing 30. The peripheral regions of the top surface (e.g., surface 37) may be used to contact the plate 20. In some embodiments, the plate 20 and housing 30 may be connected through a binder 32, which may be an adhesive, epoxy, solder, and/or any other suitable substance that can robustly attach the plate 20 and the housing 30. The binder 32 may be applied along the surface 37 of the housing 30 to allow the housing 30 to be attached to the surface 26 of the plate 20 through the binder. It should be appreciated that a small enough volume of the binder 32 may be employed, such that when the plate 20 is brought into contact with the housing 30, the binder 32 may not significantly overflow into the reservoir 31. In some embodiments, the binder 32 volume may be at least 10 μL, 20 μL, 30 μL, 50 μL, and/or any other suitable volume, such as less than or equal to 100 μL, 50 μL, 30 μL, 20 μL, 10 μL, and/or any other suitable volume. Combinations of ranges of the foregoing are also contemplated, such as between 10 and 100 μL, 20 and 100 μL, and/or any other suitable range of volumes. It should be appreciated that the present disclosure is not limited by the volume, material composition, and/or arrangement of the binder.

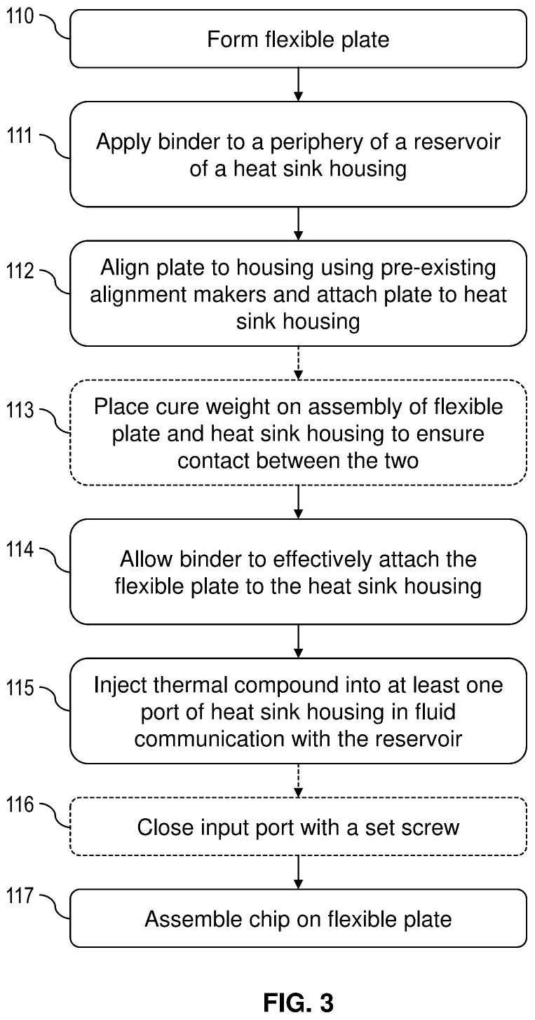

FIG. 3 shows, according to some embodiments, a flow chart for a method of assembly of a heat transfer system. In block 110, a user may form, or may be provided with, a flexible plate. As described previously, the plate may be formed with any suitable manufacturing technique to render it flexible about a central portion, such that it may elastically deform to receive a chip. In some embodiments, the plate may be formed from a metal sheet, as described in greater detail above. In block 111, the user may apply a volume of a binder (e.g., adhesive, epoxy, solder) to a periphery of a reservoir formed in a heat sink housing (see, for example, surface 37 of housing 30 in FIG. 2). In block 112, the user may employ the alignment markers (see, for example, markers 24 and surface 34 in FIG. 2) to align the plate and heat sink housing. This alignment process may take place manually (e.g., the user may visually align the two components), or an auxiliary component may be used to facilitate alignment. The user may then gently place the plate on the heat sink housing to attach the two through the binder. Optionally, as shown in block 113, the user may use a cure weight to ensure proper contact between the plate and the heat sink housing.

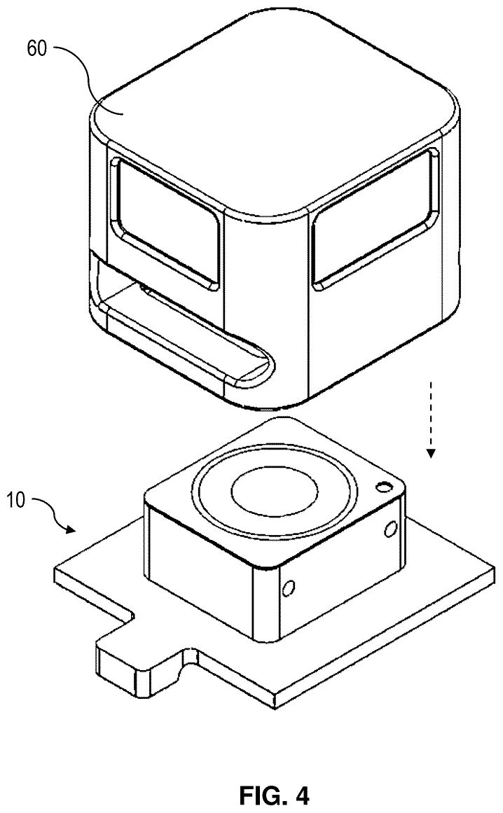

FIG. 4 shows a cure weight 60 according to some embodiments, which may be placed on top of a heat transfer system 10 to apply pressure to the plate and heat sink housing and facilitate attachment in the direction depicted by the arrow. In some embodiments, the cure weight 60 may also work to spatially align the plate and housing prior to proper adhesion between the two components. In other words, the cure weight 60 may be sized to reduce movement of the plate with respect to the housing while the binder is drying, curing, and/or any other process by which it may attach the plate and housing. It should be appreciated that the cure weight 60 may be re-usable, such that it may be removed from the system 10 after the binder has sufficiently adhered the plate and housing components. In some embodiments, the binder may rapidly attach the plate and housing components, such that a cure weight may not be necessary. For example, the binder may be a solder, which may cool immediately upon exposure to air, and may not require significant cure/drying time, when compared to an adhesive. It should be appreciated that the present disclosure is not limited by the process by which the plate is attached to the heat sink housing.

Turning back to FIG. 3, in block 114, a user may allow the binder to effectively attach the plate and the housing. In some embodiments, this process may involve thermal curing, radiative curing, light-based curing, time-based curing, drying, setting, and/or any other suitable method. As noted previously, the present disclosure is not limited by the mechanism through which the plate is attached to the heat sink housing. In block 115, a user may inject a volume of thermal compound through at least one port of the heat sink housing. In some embodiments, the at least one port may be in fluid communication with a reservoir formed in the heat sink housing, such that the thermal compound may flow into the reservoir through the port (and/or any other suitable fluidic channels). Optionally, as shown in block 116, the user may elect to close one of the ports of the reservoir with a set screw or any other suitable closure means (e.g., the port may be taped or glued shut). In block 117, the user may place a chip or associated chip interface module on the flexible plate, such that the flexible plate may elastically deform to receive the chip and conform around the chip. It should be appreciated that at this point during the assembly process, the chip, flexible plate, thermal compound, and heat sink housing (and therefore, the heat sink itself) may be in thermal communication.

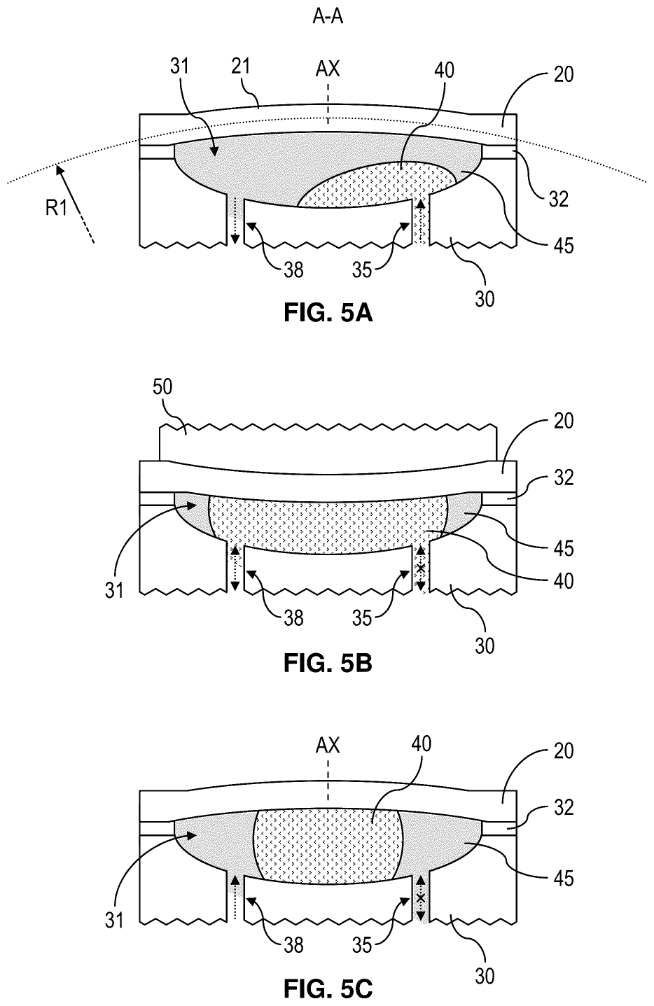

In operation, once the heat transfer system has been assembled, a chip may be installed on the system to allow a heat sink to thermally regulate the chip (e.g., cool the chip). FIGS. 5A-5C depict partial cross-sectional views of the heat transfer system in operation, according to some embodiments. In FIG. 5A, a user may be injecting a thermal compound 40 through a port 35 of a heat sink housing 30. As discussed earlier, it should be appreciated that the port 35 may be fluidically connected to a reservoir 31 of the housing 30 through one or more channels. However, FIGS. 5A-5C depict a simplified fluidic system for explanation purposes only. Accordingly, the housing 30 may include one or more channels arranged in any suitable manner to fluidically connect the reservoir 31 and the port 35.

As shown in FIGS. 5A-5C, it should be appreciated that the thermal compound 40 may not be in direct contact with an upper surface of the plate 20, where the chip 50 may be installed. This physical separation of the thermal compound 40 and chip 50 may reduce the risk of contamination of the chip 50 with the thermal compound 40. In other words, a user may not be required to apply a new volume of thermal compound to chips which may be used in the instrument. As noted previously, the heat transfer systems disclosed herein may be used to receive and release hundreds and thousands of chips in their lifetimes. Accordingly, the separation of the thermal compound and chips may render the insertion/removal process of the chips more efficient in terms of time (e.g., the user may not need to apply and remove thermal compound from each chip and/or from the instrument) and material (e.g., the same volume of thermal compound may be employed for more than one chip). It should be appreciated that although the chips and thermal compound may not be directly in contact, they may still be in thermal communication through the flexible plate, which may be thermally conductive.

As shown in FIG. 5A, injection of thermal compound 40 into reservoir 31 may displace air 45 contained within the reservoir. As discussed previously, the plate 20 and housing 30 may be connected through binder 32, such that the reservoir 31 may only be fluidically connected to the external environment through ports 35 and 38. Accordingly, to avoid pressurization of the reservoir 31, air 45 may flow out of the port 38 into the surrounding environment, as depicted by the directional dotted arrow in FIG. 5A.

It should be appreciated that at this stage of operation, the chip may not have been installed on the heat transfer system, such that the plate 20 may be in a first configuration, as shown in FIG. 5A. In some embodiments, the plate 20 (more specifically, surface 21 of the plate, as shown in FIG. 2), may have a radius R1, such that the plate may be curved out of the plane of the plate 20. As shown in FIG. 5A, this radius R1 may be sufficiently large, such that the surface 21 may only be slightly curved. In some embodiments, this curvature may provide the surface 21 with flexibility, akin to a membrane. Accordingly, upon installation of a chip 50 (and/or a chip interface module), the plate 20 may elastically deform about axis AX (e.g., deform out of plane of the surface 21) to conform about a surface of the chip 50 in a second configuration, an example of which is shown in FIG. 5B. The deformation of the plate 20 may reduce the spatial volume of the reservoir 31, such that the thermal compound 40 may be deformed or compressed in between the plate 20 and housing 30. In some embodiments, thermal compound 40 (and/or air 45) may flow out of one or more ports (e.g., port 38) to accommodate the reduction in volume of the reservoir 31. In some embodiments, the channels/ports may be positioned to reduce the likelihood of thermal compound 40 from flowing out of the reservoir 31. In some embodiments, the reservoir 31 may be substantially filled with thermal compound 40, such that elastic deformation of the plate 20 may result in thermal compound 40 flowing in and out of the port 38 (or any other suitable port and/or channel). As shown in FIG. 5B and discussed previously, in some embodiments, the port 35 may be blocked (e.g., with a set screw) to prevent fluid flow in and/or out of the port and associated fluidic channel/network. In some embodiments, the reservoir 31, ports (e.g., port 38), and thermal compound 40 may be configured to minimize the remaining volume of insulating air 45 in the reservoir 31 when the chip 50 (and/or chip interface module) is installed.

In some embodiments, as shown in FIG. 5C, when the chip 50 is removed from the system, the flexible plate 20 may elastically relax back into its original configuration (similar to FIG. 5A). Accordingly, the deformation of the plate 20 may increase the spatial volume of the reservoir 31, such that air 45 may flow into the reservoir 31 through port 38 (and/or through any other suitable port), as depicted by the arrow in FIG. 5C.

It should be appreciated that the volume of thermal compound used may be sufficient to allow the heat sink (inside the heat sink housing) to be in thermal communication with the flexible plate, regardless of the configuration of the plate. In other words, regardless of whether or not the plate has been elastically deformed about its axis (e.g., in the configuration shown in FIG. 5A or the one shown in FIG. 5B), the thermal compound may make sufficient thermal contact with both the plate and housing. In this way, the thermal circuit of the plate, thermal compound, heat sink housing, and heat sink may not be unintentionally disrupted during loading, unloading, and reloading of various-sized chips on the heat transfer system. In some embodiments, the surface adhesion between the thermal compound and the bottom surface of the plate (see surface 26 in FIG. 2) may be sufficiently strong to counteract any long-term viscous flow effects corresponding to gravity or any other external force. In some embodiments, the bottom surface of the plate may be coated with a substance to promote prolonged adhesion with the thermal compound. In other embodiments, the plate material may be selected to improve adhesion with the thermal compound.

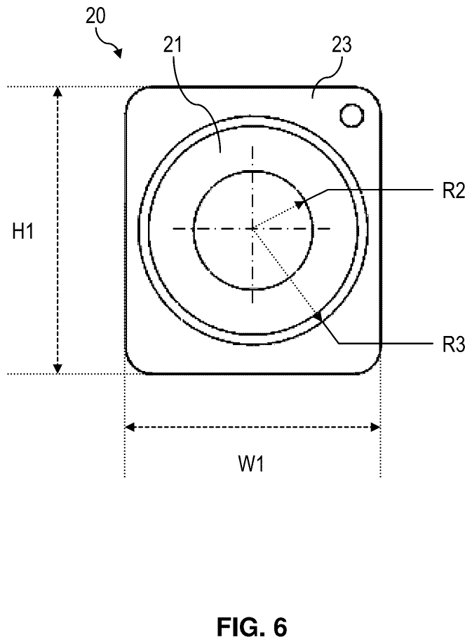

It should be appreciated that the plate may be formed of any suitable material with sufficient thermal conductivity and sized/shaped in any suitable manner to suit the intended purpose, as the present disclosure is not so limited. In an exemplary embodiment, the plate 20 has a substantially rectangular shape, as depicted in FIG. 6, to correspond to a surface of a heat sink housing. The plate 20 may have a height H1 in a first direction and a width W1 in a second direction, as well as a thickness measured normal to a plane of the plate 20. In one non-limiting embodiment, the height H1 is 17 mm and the width W1 is 16 mm. Of course, other sizes and/or shapes of the plate 20 are also contemplated, as the present disclosure is not so limited.

As shown in the cross-sectional view of FIG. 5A, in some embodiments, the plate 20 may be coined, such that it may have a radius R1 measured in the cross-section. The radius R1 may only extend along a portion of the plate 20, as shown in FIGS. 5A and 6. In some embodiments, the radius R1 may only extend along a second radius R2 (measured from axis AX) or a third radius R3 (measured from axis AX). Accordingly, the peripheral portions of the plate 20 (e.g., surface 23) may be substantially flat, normal to the axis AX. In some embodiments, radius R1 may be 150 mm. In some embodiments, radius R1 may be any suitable value, including, but not limited to, at least 50 mm, 80 mm, 90 mm, 100 mm, 120 mm, 140 mm, 150 mm, 160 mm, 180 mm, 200 mm, 250 mm, 300 mm, and/or any other suitable value. In some embodiments, radius R1 may be less than or equal to 300 mm, 250 mm, 200 mm, 180 mm, 160 mm, 150 mm, 140 mm, 120 mm, 100 mm, 90 mm, 80 mm, 50 mm, and/or any other suitable value. Combinations of the foregoing ranges are also contemplated, including between 50 mm and 300 mm, 80 mm and 250 mm, and/or any other suitable range of radii. It should be appreciated that the radius R1 may be any suitable value to render the flexible plate 20 elastically deformable about axis AX. Accordingly, depending on the material properties (e.g., elastic modulus) and geometric properties (e.g., thickness normal to the axis AX) of the plate, the radius R1 may be adjusted to ensure that the plate is sufficiently flexible about axis AX. It should be appreciated that the plate may have any suitable geometry (e.g., non-radial) to render it elastically deformable. As noted previously, the elastic deformability of the plate may have one or more benefits associated with the ability to accommodate a variety of differently sized chips, withstand hundreds and thousands of cycles of insertion/removal cycles of the chips to/from the instrument, respond to pressures exerted by supporting structures of the instrument, and/or any other benefit not previously outlined.

Similarly, radii R2 and R3 may be selected to ensure sufficient flexibility of the flexible surface 21 of the plate 20 while maintaining a suitable size of the peripheral regions (e.g., surface 23) to adhere to the heat sink housing. In one non-limiting embodiment, radius R2 is 4.2 mm and radius R3 is 8.5 mm. In some embodiments, the radius R2 may be any suitable value, including but not limited to, between 2 mm and 5 mm, 2 mm and 10 mm, 0.5 mm and 20 mm, 0.2 mm and 40 mm, and/or any other suitable size. In some embodiment, the radius R3 may be any suitable value, including, but not limited to, between 2 mm and 20 mm, 5 mm and 10 mm, 5 mm and 25 mm, 1 mm and 50 mm, and/or any other suitable size. Of course, other radial values for the plate 20 are also contemplated, as the present disclosure is not so limited. It should also be appreciated that differently shaped plates may also be employed, wherein the flexible surface 21 may not be circular or elliptical. Of course, other shapes of the plate are also contemplated, such that the present disclosure is not limited by the geometry or arrangement of any of the features described herein. It should be appreciated that the plate 20 may be any suitable shape, and may achieve flexibility with any suitable geometric arrangement.

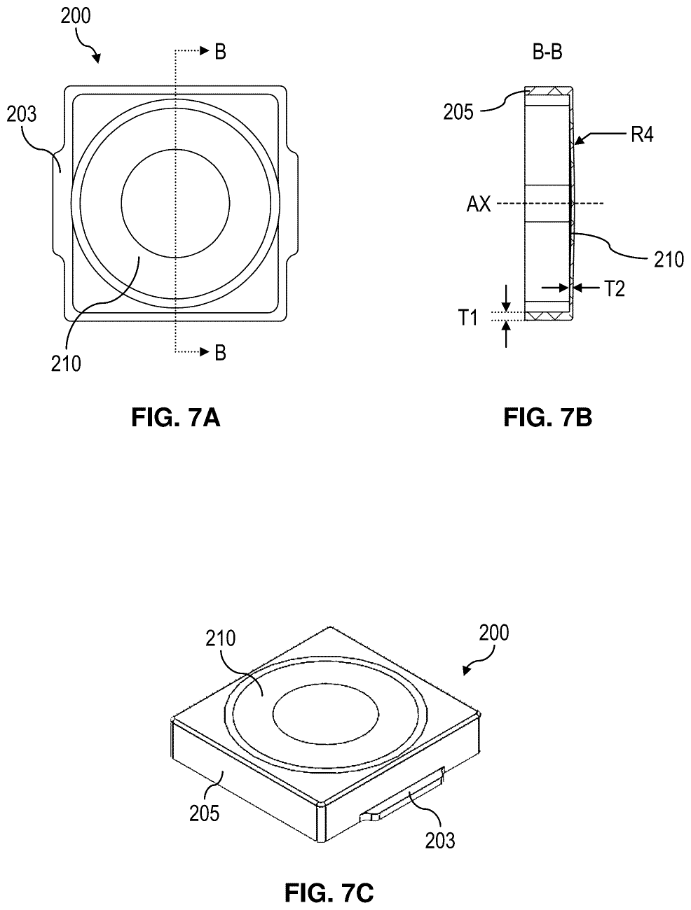

FIGS. 7A-7C depict a flexible plate 200 according to another embodiment. The plate 200 may include a flexible surface 210 similar to surface 21 shown in FIG. 2, as well as sidewalls 205 extending substantially normal to the surface 210. In some embodiments, the plate 200 may include one or more tabs 203 to facilitate attachment to a heat sink housing (e.g., tabs may engage with cutouts formed in the heat sink housing). Accordingly, to assemble a heat transfer system using plate 200, a user may apply a thermal compound on a reservoir of the heat sink housing (see reservoir 31 in FIG. 2, for example), prior to installation of the plate 200. Similar to the flexible plate described with respect to FIG. 2, plate 200 may deform to conform to a variety of differently sized chips, during insertion/removal cycles of chips, and/or in response to pressures exerted by supporting structures of the instrument. In some embodiments, air and/or thermal compound may flow out of the reservoir to adjust to a changing internal volume (when the surface 210 deforms to accept a chip, for example) through the cutouts of the heat sink housing, and/or through one or more vents formed in the plate 200.

As shown in the cross-sectional view of FIG. 7A taken along line B-B, in some embodiments, the plate 200 may have a first thickness T1, measured across the sidewalls 205, and a second thickness T2, measured across the flexible surface 210. In some embodiments, the first thickness T1 may be greater than the second thickness T2, such that the surface 210 may be more flexible than the sidewalls 205. In some embodiments, the plate 200 may be formed with a machining process (e.g., to form the sidewalls 205) as well as one or more polishing processes to form a flexible surface 210. In some embodiments, the polishing processes may yield a curved surface 210 with a radius R4, as shown in FIG. 7B. It should be appreciated that the surface 210 may be curved similarly to surface 21 shown in FIG. 2, such that the radius R4 may be sufficiently large to render a lightly curved surface 210. The radius R4 may be any suitable value to render the surface 210 elastically deformable, as shown in FIG. 2 with reference to plate 20. It should be appreciated that although a curved surface is depicted, any other suitable flexible geometry may be employed, as the present disclosure is not limited by the geometry or arrangement of the plate.

Having thus described several aspects of several embodiments of a heat transfer system, it is to be appreciated that various alterations, modifications, and improvements will readily occur to those skilled in the art. Such alterations, modifications, and improvements are intended to be part of this disclosure, and are intended to be within the spirit and scope of the invention. While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Further, though some advantages of the present invention may be indicated, it should be appreciated that not every embodiment of the invention will include every described advantage. Some embodiments may not implement any features described as advantageous. Accordingly, the foregoing description and drawings are by way of example only.

The section headings used are for organizational purposes only and are not to be construed as limiting the subject matter described in any way.

Also, the technology described may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used, should be understood to control over dictionary definitions, definitions in documents incorporated by reference, and/or ordinary meanings of the defined terms.

The indefinite articles “a” and “an,” as used in the specification and in the claims, unless clearly indicated to the contrary, should be understood to mean “at least one.”

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The term “substantially” as used herein may be construed to mean within 50% of a target value in some embodiments, within 60% of a target value in some embodiments, within 70% of a target value in some embodiments, within 75% of a target value in some embodiments, within 80% of a target value in some embodiments, within 85% of a target value in some embodiments, within 90% of a target value in some embodiments, within 95% of a target value in some embodiments, within 98% of a target value in some embodiments, within 99% of a target value in some embodiments, and within 99.5% of a target value in some embodiments. In some embodiments, the term “substantially” may equal 100% of the target value. For example, the term “substantially elastic deformation” may refer to deformation wherein at least 75% of the deformation comprises elastic deformation, with the remaining deformation being plastic or an intermediate deformation. In another example, the term “substantially elastic deformation” may refer to deformation wherein at least 99% of the deformation comprises elastic deformation.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. A heat transfer system comprising:
a plate configured to deform to receive an optoelectronic chip,
wherein the plate is configured to be in thermal communication with the optoelectronic chip at a first interface, and wherein the plate is in thermal communication with a heat sink via a thermally conductive material.

2. The heat transfer system of claim 1, wherein the plate is configured to undergo in-plane deformation.

3. The heat transfer system of claim 1, wherein the plate is configured to undergo substantially elastic deformation.

4. The heat transfer system of claim 1, wherein the plate is configured to receive the optoelectronic chip such that the optoelectronic chip does not directly contact the thermally conductive material.

5. The heat transfer system of claim 1, wherein the thermally conductive material is configured to flow with deformation of the plate.

6. The heat transfer system of claim 1, wherein the heat transfer system is configured to repeatedly and releasably receive at least two optoelectronic chips.

7. The heat transfer system of claim 6, wherein the heat transfer system is configured to receive the at least two optoelectronic chips such that the at least two optoelectronic chips do not directly contact the thermally conductive material.

8. The heat transfer system of claim 6, wherein a volume of the thermally conductive material may be used to thermally couple the plate to at least two of the at least two optoelectronic chips.

9. The heat transfer system of claim 1, wherein the thermally conductive material is configured to reduce a thermal resistance between the heat sink and the plate.

10. The heat transfer system of claim 1, wherein a thermal resistance of the first interface is less than 1 K/W.

11. The heat transfer system of claim 1, wherein the heat sink is configured to dissipate at least 5 W of heat away from the optoelectronic chip.

12. The heat transfer system of claim 1, further comprising at least one port through which the thermally conductive material may be introduced into the heat transfer system.

13. The heat transfer system of claim 1, further comprising at least one port configured to allow a fluid to flow out of the heat transfer system upon deformation of the plate.

14. The heat transfer system of claim 1, wherein the heat sink is configured to keep the optoelectronic chip below 27° C.

15. The heat transfer system of claim 1, wherein the heat sink is a thermo-electric cooler.

16. The heat transfer system of claim 1, wherein at least one surface of the plate comprises an electroless nickel plated layer on the plate.

17. The heat transfer system of claim 1, wherein the plate is configured to conform to at least one surface of the optoelectronic chip.

18. The heat transfer system of claim 1, wherein the plate is operatively coupled to the heat sink at a periphery of the plate.

19. The heat transfer system of claim 1, wherein the plate comprises at least one selected from the group of aluminum, copper, stainless steel, silver, lead, tin, gold, brass, nickel, and titanium.

20. A method of cooling an optoelectronic chip, the method comprising:
deforming a plate to receive the optoelectronic chip on a first surface of the plate, such that the plate and the optoelectronic chip are in thermal communication;
transferring to the plate heat from the optoelectronic chip; and
transferring to a thermally conductive material positioned on an opposing surface of the plate heat from the plate.

21. The method of claim 20, further comprising repeatedly deforming the plate to receive and release one or more optoelectronic chips.

22. The method of claim 20, wherein the thermally conductive material does not directly contact the optoelectronic chip.

23. The method of claim 20, wherein deforming the plate comprises substantially of elastically deforming the plate.

24. The method of claim 20, further comprising transferring heat from the thermally conductive material to a heat sink.

25. The method of claim 20, wherein deforming the plate comprises flowing the thermally conductive material within a reservoir disposed proximal to the plate.

26. The method of claim 20, further comprising dissipating at least 5 W of heat from the optoelectronic chip.

27. The method of claim 20, further comprising introducing the thermally conductive material into a reservoir through at least one port of the reservoir, the reservoir disposed proximal to the plate.

28. The method of claim 20, wherein deforming the plate comprises flowing air through at least one port of a reservoir, the reservoir disposed proximal to the plate.

29. The method of claim 20, further comprising maintaining the optoelectronic chip below 27° C.

30. The method of claim 24, wherein the heat sink is a thermo-electric cooler.

31. The method of claim 20, further comprising forming an electroless nickel plated layer on at least the opposing surface.

32. The method of claim 20, wherein deforming the plate comprises conforming the first surface to at least one surface of the optoelectronic chip.

33. The method of claim 24, further comprising operatively coupling the plate to the heat sink at a periphery of the opposing surface.

34. A method of manufacturing a heat transfer system, the method comprising:
coupling at least a portion of a first surface of a plate to at least a portion of a first surface of a housing to form a reservoir therebetween; and
introducing a thermally conductive material into the reservoir through at least one port formed in the housing;
wherein the thermally conductive material is in thermal communication with the plate and the housing, and wherein the housing is in thermal communication with a heat sink.

35. The method of claim 34, wherein at least a portion of the plate is configured to deform along an in-plane direction to receive an optoelectronic chip.

36. The method of claim 35, wherein the at least a portion of the plate is configured to deform substantially elastically.

37. The method of claim 34, wherein a thermal circuit comprising the plate, the thermally conductive material, the housing, and the heat sink is configured to cool an optoelectronic chip.

38. The method of claim 34, further comprising flowing air out of at least a second port formed in the housing.

39. The method of claim 34, further comprising sealing the at least one port.

40. The method of claim 34, further comprising coupling the at least a portion of the plate and the at least a portion of the housing with adhesives and/or soldering.

41. The heat transfer system of claim 34, wherein the plate comprises at least one selected from the group of aluminum, copper, stainless steel, silver, lead, tin, gold, brass, nickel, and titanium.

42. A heat transfer system comprising:
a thermal interface configured to undergo substantially elastic deformation to receive a chip,
wherein the thermal interface is configured to be in thermal communication with the chip, and wherein the thermal interface is configured to repeatedly receive and release one or more chips.

43. The heat transfer system of claim 42, further comprising a thermally conductive material configured to conduct heat from the chip.

44. The heat transfer system of claim 43, wherein the thermally conductive material does not directly contact the chip.

45. The heat transfer system of claim 42, wherein a thermal resistance of the thermal interface is less than 1 K/W.

46. The heat transfer system of claim 42, wherein the thermal interface is in thermal communication with a heat sink.

47. The heat transfer system of claim 46, wherein the heat sink is configured to dissipate at least 5 W of heat away from the chip.

48. The heat transfer system of claim 42, wherein the thermal interface comprises at least one selected from the group of aluminum, copper, stainless steel, silver, lead, tin, gold, brass, nickel, and titanium.

49. The heat transfer system of claim 42, wherein the chip is an optoelectronic chip.
